(19) United States
(12) Patent Application Publication
(10) Pub. No.: US 2005/0033354 A1
(43) Pub. Date: Feb. 10, 2005

(54) Endoscopic surgical instrument

(75) Inventors: Richard Montalvo, Miami, FL (US); Ceasar Escobar, Miami, FL (US); Curt Willem, Miami, FL (US)

Correspondence Address: FINNEGAN, HENDERSON, FARABOW, GARRETT & DUNNER;LLP, 1300 I STREET, NW, WASHINGTON, DC, 20005, US

(73) Assignee: SCIMED Life Systems, Inc., Maple Grove, MN (US)

(21) Appl. No.: 10/911,487

(22) Filed: Aug. 5, 2004

Related U.S. Application Data
(63) Continuation of application No. 09/988,125, filed on Nov. 19, 2001, now abandoned.

(51) Int. Cl. A61B 17/28

(52) U.S. Cl. 606/205

(57) ABSTRACT

An endoscopic surgical instrument has a mechanism for connecting an end effector assembly of to an elongate catheter of the instrument. The surgical instrument includes a tubular member having a distal end and a proximal end. A handle attaches to the proximal end of the tubular member. An end effector assembly has an end effector and an attachment portion for releasably attaching to the distal end of the tubular member. The attachment portion has a protrusion for resisting detachment of the attachment portion and the tubular member. An actuator connects to the handle and the end effector assembly for actuating the end effector.

### BACKGROUND OF THE INVENTION

1. Field of the Intention

The present invention relates to a surgical instrument. More particularly, this invention relates to an endoscopic surgical instrument for performing an operation within a body cavity in conjunction with an endoscope, and, even more particularly, to a mechanism for connecting an end effector assembly of such an instrument to an elongate catheter of the instrument.

2. Background of the Related Art

Various surgical instruments may be used in connection with an endoscope for performing a number of operations at a site deep within a patient's body cavity. One such instrument, a biopsy forceps device, samples tissue from a body cavity with minimal intervention and discomfort to patients. Typically, a biopsy forceps device, like other endoscopic instruments, has a long flexible tubular member of small diameter for insertion into a lumen of the endoscope. An end effector assembly, such as a distal forceps assembly, attaches at a distal end of the tubular member, and a handle attaches at a proximal end of the tubular member. An actuator, such as a pull wire, connects the end effector assembly and the handle through the tubular member. A biopsy forceps assembly, for example, may include mating jaws actuated by the handle to sample a body tissue. For the end effector assembly to reach a site deep in a body cavity, the tubular member is sufficiently long and flexible to follow a long, winding path of the body cavity.

A typical endoscopic device has a tubular member made of a tightly-wound helical coil. The helical coil is usually made of stainless steel. The helical-coil tubular member is relatively expensive and complex to manufacture. Thus, the helical-coil tubular member is less suitable for disposable use.

The end effector assembly of a typical endoscopic device, for example a distal forceps assembly of a typical biopsy forceps device, attaches to one end of the helical-coil tubular member by a crimping process. This crimping process, however, subjects the distal assembly to accidental detachment from the helical-coil tubular member. Detachment within a body cavity results in significant inconvenience.

### SUMMARY OF THE INVENTION

The advantages and purposes of the invention will be set forth in part in the description which follows, and in part will be apparent from the description, or may be learned by practice of the invention. The advantages and purposes of the invention will be realized and attained by the elements and combinations particularly pointed out in the appended claims.

To attain the advantages and in accordance with the purposes of the invention, as embodied and broadly described herein, the invention includes a surgical instrument including a tubular member having a distal end and a proximal end, a handle attached to the proximal end of the tubular member, and an end effector assembly having an end effector and an attachment portion for releasably attaching to the distal end of the tubular member. The attachment portion has a protrusion for resisting detachment of the attachment portion and the tubular member. An actuator connects to the handle and the end effector assembly for actuating the end effector.

In accordance with another aspect of the invention, the tubular member has a hollow, coil-less structure.

In yet another aspect of the invention, an end effector assembly of a surgical instrument having an elongate tubular member includes an end effector. An attachment portion connects to the end effector and is configured to attach releasably to the tubular member. The attachment portion has a protrusion for resisting detachment of the attachment portion and the tubular member.

It is to be understood that both the foregoing general description and the following detailed description are exemplary and explanatory only and are not restrictive of the invention, as claimed.

### BRIEF DESCRIPTION OF THE DRAWINGS

The accompanying drawings, which are incorporated in and constitute a part of this specification, illustrate several embodiments of the invention and together with the description, serve to explain the principles of the invention. In the drawings,

FIG. 1 is a side and partially cross-sectional view of a first embodiment of an endoscopic surgical instrument according to the present invention;

FIG. 2 is a cross-sectional view of a multi-lumen type tubular member in an axial direction according to the present invention;

FIG. 3 is a cross-sectional view of the tubular member shown in FIG. 2 in a lateral direction;

FIG. 4 is a partial side view of the distal end of the instrument shown in FIG. 1;

FIG. 5 is a perspective view of the end effector assembly shown in FIG. 1;

FIG. 6 is a partial perspective view of a second embodiment of an endoscopic surgical instrument according to the present invention;

FIG. 7 is a perspective view of the end effector assembly shown in FIG. 6;

FIG. 8 is a side view of a third embodiment of an endoscopic surgical instrument according to the present invention;

FIG. 9 is a side view of the end effector assembly shown in FIG. 8; and

FIG. 10 is a perspective view of an end effector support member shown in FIG. 8.

### DESCRIPTION OF THE PREFERRED EMBODIMENTS

Reference will now be made in detail to the present preferred embodiments of the invention, examples of which are illustrated in the accompanying drawings. Wherever possible, the same reference numbers will be used throughout the drawings to refer to the same or like parts.

The present invention is related to an endoscopic instrument for use in conjunction with an endoscope for performing an operation within a body cavity of a patient. The endoscopic instrument generally includes an elongate, flexible tubular member having a distal end and a proximal end. A handle attaches to the proximal end of the tubular member. An end effector assembly, such as, for example a distal biopsy forceps assembly, attaches to the distal end of the tubular member. An actuator is connected to the handle and end effectors, for example jaws, through the flexible tubular member for actuating the end effectors.

The present invention more particularly resides in the mechanism for connecting the elongate tubular member to the end effector assembly. The end effector assembly has at least one end effector and an attachment portion for releasably attaching the end effector assembly to the distal end of the tubular member. The attachment portion preferably includes at least one protrusion that projects toward the tubular member to prevent accidental, unintended detachment of the end effector assembly from the tubular member during use. The attachment portion, however, preferably does not permanently attach the end effector assembly to the tubular member. In other words, when the surgical instrument is not in use, a sufficient force may detach the end effector assembly from the tubular member so that the user, for example, may dispose of the end effector assembly. In this way, the tubular member and the end effector assembly can be releasably and securely connected. Moreover, the present invention provides for a unique tubular member of a continuous, hollow and coil-less structure that is relatively easy to manufacture.

The present invention will be shown and described in connection with a biopsy forceps device and a distal forceps assembly having a pair of jaws as end effectors. However, it is to be recognized that the inventive connection between the end effector assembly and the tubular member, and the structure of the tubular member, are suitable for other types of endoscopic, laparoscopic, or other instruments and other types of end effector assemblies and end effectors, such as graspers, cutters, or other devices known in the art. The biopsy forceps device shown and described is exemplary only.

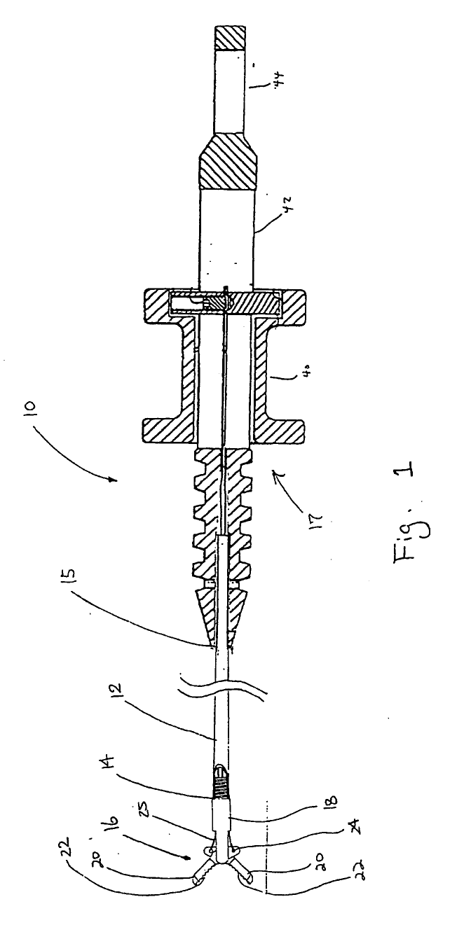

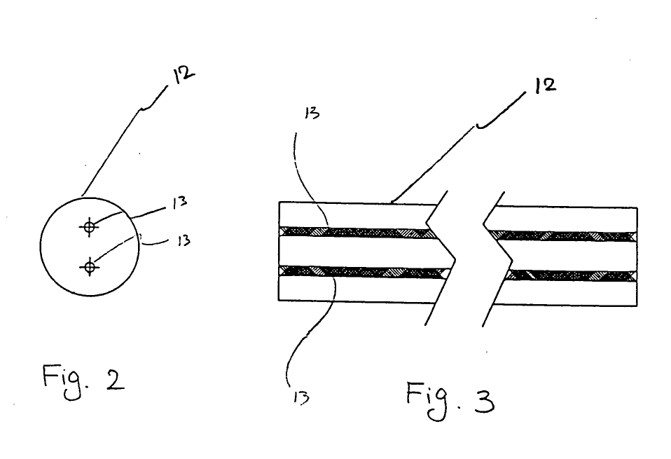

In a first embodiment of the present invention shown in FIG. 1, a biopsy forceps device 10 has a flexible tubular member 12 having a distal end 14 and a proximal end 15. During an operation on a patient to obtain a biopsy, distal end 14 travels through a lumen of an endoscope and the patient's body cavity. Proximal end 15 of tubular member 12 remains outside the body cavity. Tubular member 12 should be made of a material that has sufficient stiffness, elasticity, and maneuverability to sustain, for example, bending and shear forces incurred during a biopsy operation. Preferably, tubular member 12 is made of a nylon resin or any other suitable plastic materials of similar characteristics. Tubular member 12 preferably has a hollow, coil-less structure, and is preferably manufactured by an extrusion process. Tubular member 12, moreover, preferably has a diameter small enough to fit through a lumen of an endoscope and should be free of kinks or any excessive protrusions or bumps for ease of passage through the endoscope. Tubular member 12 can be of a single lumen type as shown in FIG. 1 or have multiple lumens 13 as shown in FIGS. 2 and 3.

A handle 17 is attached to the proximal end of tubular member 12. Handle 17 is used to control or actuate a distal biopsy forceps assembly 16 within a body cavity. As shown in FIG. 1, handle 17 is a conventional spool and shaft actuator having a spool 40 surrounding a shaft 42 having a thumb ring 44. An actuation wire (described later) attaches to handle 17 in a manner well known in the art. A typical spool and shaft actuator is described in detail in U.S. Pat. No. 5,553,624, which is incorporated herein by reference. Other types of handles known in the art also can be used in combination with the tubular member and distal attachment mechanism of this invention. The handle shown and described is exemplary only.

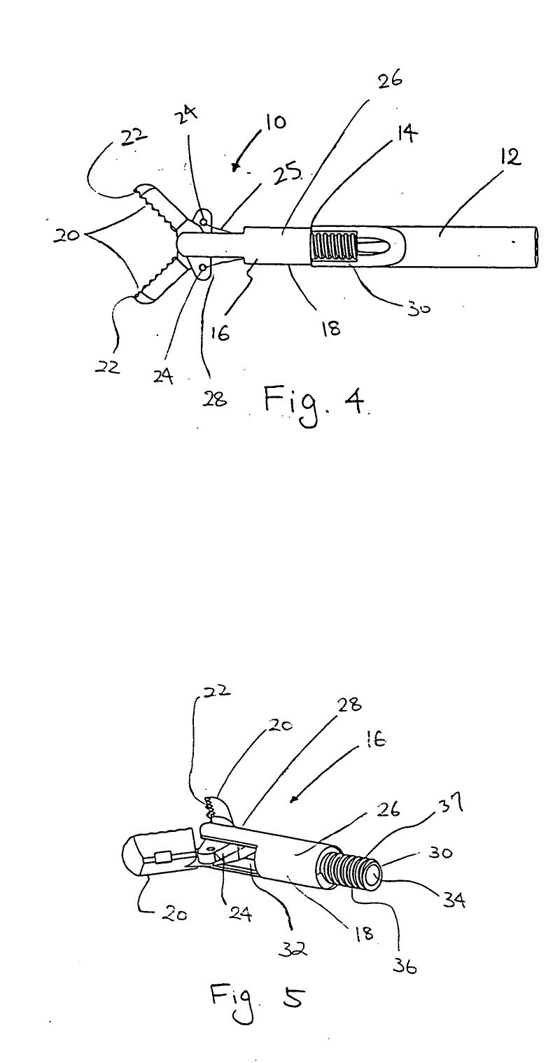

As illustrated in FIGS. 1, 4, and 5, an end effector assembly, for example a distal biopsy forceps assembly 16, has a jaw support member 18 and two mating forceps jaws 20 pivotally connected to jaw support member 18. Biopsy forceps assembly 16 can be made of any high-impact resistant material. Preferably, biopsy forceps assembly 16 is injection molded with a high-impact plastic material or made of both metal and plastic.

Forceps jaws 20 are pivotally connected to jaw support member 18 such that they can be readily opened and closed to sample a tissue within a body cavity. Forceps jaws 20 may be pivotally connected to jaw support member 18 with a pin that extends through one side of support member 18 to the other side, or by any other suitable method known in the art. Each forceps jaw 20 has a generally hemispherical shape and has teeth 22 on a peripheral edge. Teeth 22 of each forceps jaw 20 inwardly face teeth 22 of the other forceps jaw 20 such that upon closing forceps jaws 20, teeth 22 of forceps jaws 20 mate. Teeth 22 should have a suitable shape to readily sample a body tissue. Typically, each forceps jaw 20 has an aperture 24 at a proximal end of jaw 20 for connecting with a flexible elongate actuator 25.

Flexible elongate actuator 25 connects to the handle at its proximal end, extends through elongate tubular member 12, and connects to forceps jaws 20 at a distal end of actuator 25. Preferably, actuator 25 is a pair of pull wires, as are well known in the art, that can be made of any suitable material such as steel. Flexible elongate actuator 25 is sized to fit inside tubular member 12 and extend from the handle to forceps jaw 20 through tubular member 12. Flexible elongate actuator 25 actuates jaws 20 of biopsy forceps assembly to open and close jaws 20. The flexible elongate member should also be able to withstand bending and tensile forces when actuating jaws 20.

FIGS. 1, 4, and 5 illustrate one preferred embodiment of jaw support member 18. Jaw support member 18 is preferably a clevis capable of supporting forceps jaws 20. Jaw support member 18 has a construction suitable for disposable use. Jaw support member 18 has a base portion 26, two arms 28 extending from base portion 26 in the axial direction, and an annular attachment portion 30. Preferably, base portion 26, arms 28, and annular attachment portion 30 are integrally molded. Base portion 26 preferably includes a passageway so that the flexible elongate member can extend from the handle to forceps jaws 20 through base portion 26. Arms 28 define a slot 32 there between, and forceps jaws 20 are pivotally connected to arms 28 in slot 32 such that they can open and close without obstruction by jaw support member 18. Attachment portion 30 is configured to attach releasably to distal end 14 of tubular member 12.

In one preferred embodiment shown in FIGS. 1, 4, and 5, attachment portion 30 has an inner wall 34 and an outer wall 36. As shown in FIG. 3 in detail, outer wall 36 is threaded to define at least one protrusion 37 that projects from outer wall 36. In this embodiment, protrusion 37 is a threaded surface for providing a resistive force between outer wall 36 and the inside of tubular member 12. Attachment portion 30 inserts into flexible tubular member 12 and flexible tubular member 12 covers threaded outer wall 36 of attachment portion 30. Protrusion (thread) 37 may dig into the inner surface of tubular member 12 and counter a pull force tending to separate tubular member 12 and end effector assembly 16, avoiding accidental detachment within a body cavity, or protrusion 37 simply may provide a frictional force opposing a force tending to separate tubular member 12 and end effector assembly 16 during endoscopic operation. Attachment portion 30, however, may be detached from tubular member 12 by a sufficiently stronger pull force for disposal of end effector assembly 16.

Attachment portion 30 may be attached to tubular member 12 by ultrasonic welding. In such an arrangement, attachment portion 30 cannot be detached from tubular member 12.

The threaded surface of attachment portion 30 can have various configurations, such as different pitch and thread shape, and provides a secure attachment of attachment portion 30 to distal end 14 of tubular member 12 so that biopsy forceps assembly 16 and tubular member 12 do not disengage accidentally during a biopsy operation. Biopsy forceps assembly 16 having threaded attachment portion 30, moreover, can be manufactured at relatively low cost.

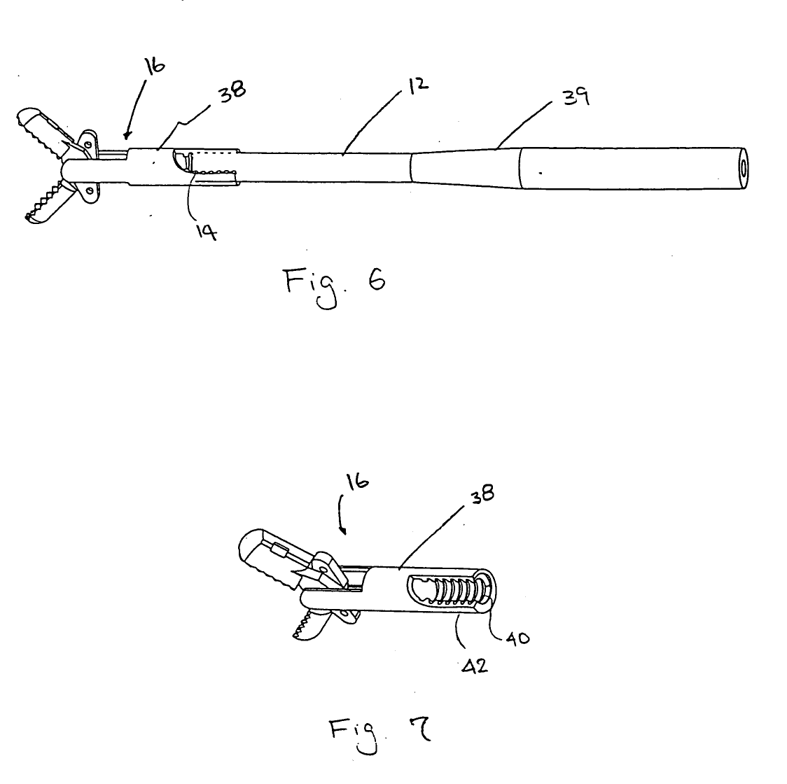

In another preferred embodiment shown in FIGS. 6 and 7, an annular attachment portion 38 has an inner wall 40 with at least one protrusion 42 for releasably attaching to distal end 14 of tubular member 12. As illustrated in FIG. 6, biopsy forceps assembly 16 attaches to distal end 14 of flexible tubular member 12 such that flexible tubular member 12 inserts into annular attachment portion 38 of biopsy forceps assembly 16. Inner wall 40 has protrusion 42 for providing a resistive force between inner wall 40 and tubular member 12. When assembled, distal end 14 of flexible tubular member 12 is covered by threaded inner wall 40 of annular attachment portion 38. Similar to the embodiment shown in FIGS. 1, 4, and 5, the threads on inner wall 40 of attachment portion 38 can have various configurations to provide sufficient force to prevent attachment portion 38 and tubular member 12 from accidental disengagement during a biopsy operation. In this embodiment, tubular member 12 has a step-down portion 39. Step-down portion 39 reduces the diameter of tubular member 12 at distal end 14 to fit into attachment portion 30.

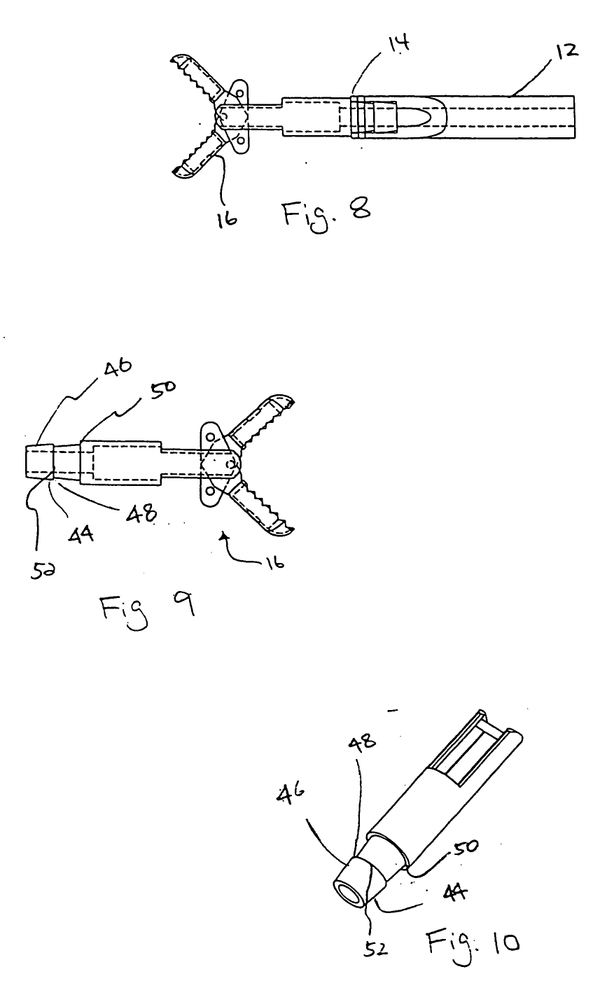

FIGS. 8-10 illustrate another preferred embodiment of an attachment portion 44. Attachment portion 44 has an outer wall 46 having a protrusion 48 that resists a pull force so that the forceps assembly may releasably attach to distal end 14 of tubular member 12. Protrusion 48 is a barbed or stepped portion defined by two different diameters of attachment portion 44. As shown in FIG. 8, biopsy forceps assembly 16 attaches to distal end 14 of flexible tubular member 12. In this configuration, attachment portion 44 inserts into flexible tubular member 12 and tubular member 12 covers barbed outer wall 46 of attachment portion 44. Preferably, attachment portion 44 has a first step 50 for distal end 14 of tubular member 12 to meet, and a second step 52 defining protrusion 48. Protrusion 48 is configured to provide a resistive force to securely keep tubular member 12 attached to attachment portion 30 during use. In another embodiment, attachment portion 30 may be crimped to tubular member 12.

Several experimental tests were performed on each embodiment of this invention. First, a series of pull tests were performed to measure a pull force required to remove the distal biopsy forceps assembly from the tubular member. The forceps assembly first was attached to the tubular member. Then, the pull force required to detach the forceps assembly from the tubular member was measured. These tests showed that the distal biopsy forceps assembly of this invention provided sufficient resistance to pull forces typically encountered during endoscopic use. The tests further showed that the forceps assembly may be detached from the tubular member by sufficiently greater forces so that the forceps may be disposed of after use.

Various other typical performance tests were performed. For example, a scope passage test determined that the biopsy forceps device can be smoothly inserted in and extracted from an endoscope lumen without causing any damage to the endoscope. A typical actuation test determined that the forceps assembly can be actuated while the tubular member was bent approximately 90 degrees. Loop tests determined that the biopsy forceps device functions after being looped in a figure eight configuration, and plastic memory tests determined that the biopsy forceps device retains curvature imparted while looped and stored for a period of time. Eraser biting tests determined that the biopsy forceps device produced sufficient bites. Each embodiment of a biopsy forceps device described herein satisfied these tests.

It will be apparent to those skilled in the art that various modifications and variations can be made in the endoscopic surgical instrument of the present invention and in construction of this endoscopic surgical instrument without departing from the scope or spirit of the invention.

Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

1. A surgical instrument, comprising:
a flexible tubular member having a distal end and a proximal end;
a handle attached to the proximal end of the flexible tubular member;
an end effector assembly having an end effector and an attachment portion for releasably attaching to the distal end of the flexible tubular member, the attachment portion having a first step portion and a second step portion facing the first step portion, the first step portion abutting an end of the flexible tubular member and the second step portion contacting an inner surface of the flexible tubular member for resisting detachment of the attachment portion and the flexible tubular member; and
an actuator connected to the handle and the end effector assembly for actuating the end effector.

2. The surgical instrument of claim 1, wherein the tubular member has a hollow, coil-less structure.

3. (Canceled)

4. The surgical instrument of claim 1, wherein the attachment portion has an inner wall and an outer wall having the first step portion and the second step portion.

5. The surgical instrument of claim 4, wherein the end effector assembly is attached to the distal end of the flexible tubular member such that the attachment portion is inserted into the flexible tubular member and the flexible tubular member covers the outer wall of the attachment portion.

6-11. (Canceled)

12. The surgical instrument of claim 1, wherein the end effector assembly is a biopsy forceps assembly having a jaw support member, and the end effector is a pair of jaws hinged to the jaw support member.

13. An end effector assembly of a surgical instrument having a flexible elongate tubular member, the end effector assembly comprising:
an end effector; and
an attachment portion connected to the end effector and configured to attach releasably to the flexible elongate tubular member, the attachment portion having a first step portion and a second step portion facing the first step portion, the first step portion capable of abutting an end of the flexible elongate tubular member and the second step portion capable of contacting an inner surface of the flexible elongate tubular member for resisting detachment of the attachment portion and the tubular member.

14. The surgical instrument of claim 13, wherein the attachment portion has an inner wall and an outer wall having the first step portion and the second step portion.

15. The surgical instrument of claim 14, wherein the attachment portion is configured to be inserted into the flexible elongate tubular member.

16-20. (Canceled)

21. The surgical instrument of claim 1, wherein the end effector assembly defines a passageway extending therethrough.

22. The surgical instrument of claim 21, wherein the attachment portion defines the passageway.

23. The surgical instrument of claim 1, wherein the actuator is a pair of pull wires.

24. The surgical instrument of claim 21, wherein the actuator is received through the passageway of the end effector assembly and extends to the proximal end of the tubular member.

25. The surgical instrument of claim 1, wherein the actuator is a flexible elongate actuator configured to connect to a proximal end of the end effector for actuating the end effector.

26. The surgical instrument of claim 13, wherein the end effector assembly defines a passageway therethrough.

27. The surgical instrument of claim 27, wherein the attachment portion defines the passageway.
